SEQUENCE LISTING (1 sequence)

SEQ ID NO: 1 - DNA, 1538 bp, Arabidopsis thaliana
aaaccactctgcttcctcttcctctgagaaatcaaatcactcacactccaaaaaaaaatctaaactttctcagagtttaatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgagagagagagctttgccttctagtttgaatttctatttcttccgcttcttcttcttttttttcttttgttgggttctgcttagggtttgtatttcagtttcagggcttgttcgttggttctgaataatcaatgtctttgccccttttctaatgctccaagttcagat

(19) United States
(12) Patent Application Publication
(10) Pub. No.: US 2009/0061492 A1
(43) Pub. Date: Mar. 5, 2009

(54) Method for producing biodiesel

(75) Inventors: Christoph Benning, East Lansing, MI (US); J. Michael Younger, Holt, MI (US)

Correspondence Address: Peter G. Carroll;MEDLEN & CARROLL, LLP, Suite 350, 101 Howard Street, San Francisco, CA, 94105, US

(73) Assignee: The Board of Trustees For Michigan State University System

(21) Appl. No.: 11/985,250

(22) Filed: Nov. 14, 2007

Related U.S. Application Data
(60) Provisional application No. 60/859,217, filed on Nov. 15, 2006.

(51) Int. Cl. C12P 7/64; A01H 5/00

(52) U.S. Cl. 435/134; 800/298; 800/320.1; 800/312; 800/322

(57) ABSTRACT

The present invention relates to the production of biodiesel. In particular, the present invention provides systems and methods for fermenting biomass materials with transgenic plant materials expressing the WRI1 transcription factor. In preferred embodiments, WRI1 is expressed in canola. The transgenic canola plants are fermented with a biomass source so that oil is produced using carbohydrates from the biomass source as an energy source.

### FIELD OF THE INVENTION

The present invention relates to the production of biodiesel. In particular, the present invention provides systems and methods for fermenting biomass materials with transgenic plant materials expressing the WRI1 transcription factor.

### BACKGROUND OF THE INVENTION

Biodiesel is the name of a clean burning alternative fuel, produced from domestic, renewable resources. Biodiesel contains no petroleum, but it can be blended at any level with petroleum diesel to create a biodiesel blend. It can be used in compression-ignition (diesel) engines with little or no modifications. Biodiesel is simple to use, biodegradable, nontoxic, and essentially free of sulfur and aromatics. Biodiesel is defined as mono-alkyl esters of long chain fatty acids derived from vegetable oils or animal fats which conform to ASTM D6751 specifications for use in diesel engines. Biodiesel refers to the pure fuel before blending with diesel fuel. Biodiesel blends are denoted as, “BXX” with “XX” representing the percentage of biodiesel contained in the blend (ie: B20 is 20% biodiesel, 80% petroleum diesel).

Biodiesel is made through a chemical process called transesterification whereby the glycerin is separated from the fat or vegetable oil. The process leaves behind two products—methyl esters (the chemical name for biodiesel) and glycerin (a valuable byproduct usually sold to be used in soaps and other products).

Fuel-grade biodiesel must be produced to strict industry specifications (ASTM D6751) in order to insure proper performance. Biodiesel is the only alternative fuel to have fully completed the health effects testing requirements of the 1990 Clean Air Act Amendments. Biodiesel that meets ASTM D6751 and is legally registered with the Environmental Protection Agency is a legal motor fuel for sale and distribution. Raw vegetable oil cannot meet biodiesel fuel specifications, it is not registered with the EPA, and it is not a legal motor fuel.

While biodiesel is an attractive alternative fuel, the large-scale production of biodiesel from renewable plant resources faces several limitations. In particular, current oilseed crops have low yields of oil per acre. This means the production of biodiesel from oilseed crops is not attractive due to low efficiency and expense. What is needed in the art is a way to produce higher amounts of plant oil per acre.

### SUMMARY OF THE INVENTION

The present invention relates to the production of biodiesel. In particular, the present invention provides systems and methods for fermenting biomass materials with transgenic plant materials expressing the WRI1 transcription factor. Accordingly, the present invention provides methods comprising: a) providing: i) first plant material from a plant comprising an exogenous WRI1 gene; ii) lignocellulosic plant material from a second plant; b) contacting the first plant material with the lignocellulosic plant material under conditions such that triacylglycerols are produced by the first plant material. In some embodiments, the first plant material is selected from the group consisting of canola, corn, soybean, sunflower and safflower plant material. In further embodiments, the first plant material is selected from the group consisting of seeds, leaves, germinated seeds, seedlings and combinations thereof. In some embodiments, the lignocellulosic plant material is selected from the group consisting of perennial grass, annual grass, perennial woody plants, and crop residue. In further embodiments, the lignocellulosic plant material is treated to hydrolyze cellulose and/or hemicellulose contained in the material. In some preferred embodiments, the lignocellulosic material is treated by a method selected from the group consisting of chemical and enzymatic treatment. In further preferred embodiments, the WRI1 gene is at least 70% identical to SEQ ID NO:1. In some embodiments, the WRI1 gene is operably linked to a promoter selected from the group consisting of 35S CMV promoter, Universal Seed Promotor, 2S Seed Storage Protein Promoter, Cruciferin promoter, and vicilin promoter. In some embodiments, the methods further comprise the step of extracting the triacylglycerols from the first plant material. In some embodiments, the methods further comprise the step of refining the triacylglycerols. In some preferred embodiments, the lignocellulosic material is pretreated prior to the chemical or enzymatic treatment.

In some embodiments, the present invention provides methods comprising: a) providing: i) first plant material from a first plant comprising an exogenous WRI1 gene (cDNA);

ii) lignocellulosic plant material from a second plant; b) treating the lignocellulosic plant material to hydrolyze cellulose and hemicellulose to provide hydrolyzed lignocellulosic plant material; c) contacting the first plant material with the hydrolyzed lignocellulosic plant material under conditions such that triacylglycerols are produced by the first plant material; and d) extracting the triacylglycerols from the first plant material.

In further embodiments, the present invention provides a feedstock for a culture process comprising first plant material comprising an exogenous WRI1 gene (cDNA) and hydrolyzed lignocellulosic plant material. In some embodiments, the first plant material is selected from the group consisting of canola, corn, soybean, sunflower and safflower plant material. In some embodiments, the first plant material is selected from the group consisting of seeds, leaves, germinated seeds, seedlings and combinations thereof. In further preferred embodiments, the hydrolyzed lignocellulosic plant material is selected from the group consisting of hydrolyzed perennial grass, annual grass, perennial woody plants, and crop residue. In some preferred embodiments, the WRI1 gene is at least 70% identical to SEQ ID NO:1. In further preferred embodiments, the WRI1 gene is operably linked to a promoter selected from the group consisting of 35S CMV promoter, Universal Seed Promotor, 2S Seed Storage Protein Promotor, Cruciferin promoter, and vicilin promoter.

### DESCRIPTION OF THE FIGURES

FIG. 1 depicts a biodiesel production scheme using a seedling fermentation process.

FIG. 2 provides the sequence if WRI1.

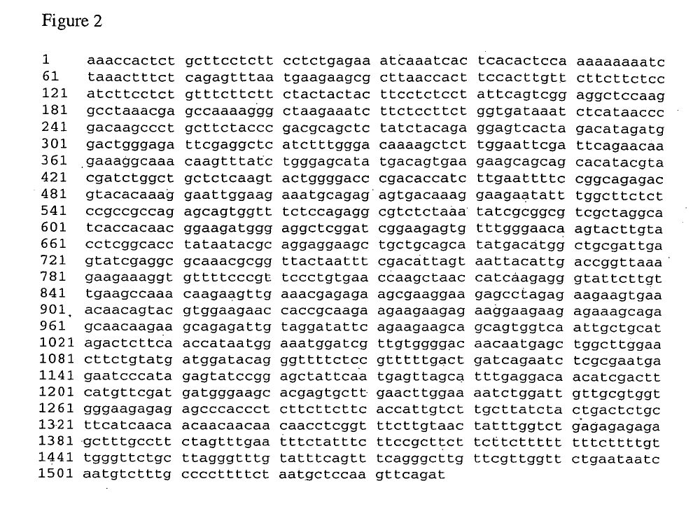

### DEFINITIONS

As used herein, the term “plant” is used in it broadest sense. It includes, but is not limited to, any species of grass (e.g. turf grass), ornamental or decorative, crop or cereal (e.g. maize, soybean), fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

As used herein, the term “plant part” as used herein refers to a plant structure or a plant tissue, for example, pollen, an ovule, a tissue, a pod, a seed, a leaf and a cell. Plant parts may comprise one or more of a tiller, plug, rhizome, sprig, stolen, meristem, crown, and the like. In some embodiments of the present invention transgenic plants are crop plants. The terms “crop” and “crop plant” is used herein its broadest sense. The term includes, but is not limited to, any species of plant or alga edible by humans or used as a feed for animals or fish or marine animals, or consumed by humans, or used by humans (natural pesticides), or viewed by humans (flowers) or any plant or alga used in industry or commerce or education. Indeed, a variety of crop plants are contemplated, including but not limited to soybean, barley, sorghum, rice, corn, wheat, tomato, potato, pepper, onions, Arabidopsis sp., melons, cotton, turf grass, sunflower, herbs and trees.

As used herein, the term “plant material” includes, plants, plant tissues and plant parts including, but not limited to, seeds, germinated seeds, and seedlings.

As used herein, the term “biomass” refers to living and recently living biological material which can be used in an industrial energy extraction process.

As used herein, the term “lignocellulosic biomass material” refers to biomass materials comprising cellulose, hemicellulose, and lignin.

As used herein, the term “saccharization” refers to the process of hydrolyzing lignocellulosic biomass material to produce sugars such as glucose, fructose, sucrose, mannose, maltose, galactose, and xylose.

As used herein, the term “WRI1 gene” refers to a gene having a nucleic acid sequence corresponding to SEQ ID NO:1 and nucleic acid sequences that are least 60% identical to SEQ ID NO:1.

As used herein, the term “transgenic” when used in reference to a plant or leaf or fruit or seed for example a “transgenic plant,” transgenic leaf,” “transgenic fruit,” “transgenic seed,” or a “transgenic host cell” refers to a plant or leaf or fruit or seed that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

As used herein, the term “transgene” refers to a foreign gene that is placed into an organism or host cell by the process of transfection. The term “foreign gene” or heterologous gene refers to any nucleic acid (e.g., gene sequence) that is introduced into the genome of an organism or tissue of an organism or a host cell by experimental manipulations, such as those described herein, and may include gene sequences found in that organism so long as the introduced gene does not reside in the same location, as does the naturally occurring gene.

As used herein, the terms “transformants” and “transformed cells” include the primary transformed cell and cultures derived from that cell without regard to the number of transfers. Resulting progeny may not be precisely identical in DNA content, due to deliberate or inadvertent mutations. Mutant progeny that have the same functionality as screened for in the originally transformed cell are included in the definition of transformants. The term “Agrobacterium” refers to a soil-borne, Gram-negative, rod-shaped phytopathogenic bacterium that causes crown gall. Agrobacterium is a representative genus of a soil-borne, Gram-negative, rod-shaped phytopathogenic bacterium family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines are a convenient source of control elements for chimeric expression cassettes. Agrobacterium tumefaciens causes crown gall disease by transferring some of its DNA to the plant host. The transferred DNA (T-DNA) is stably integrated into the plant genome, where its expression leads to the synthesis of plant hormones and thus to the tumorous growth of the cells. A putative macromolecular complex forms in the process of T-DNA transfer out of the bacterial cell into the plant cell.

The term “Agrobacterium” includes, but is not limited to, the strains Agrobacterium tumefaciens, (which typically causes crown gall in infected plants), and Agrobacterium rhizogens (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain GV3101, LBA4301, C58, A208, etc.) are referred to as “nopaline-type” Agrobacteria; Agrobacterium strains which cause production of octopine (e.g., strain LBA4404, Ach5, B6, etc.) are referred to as “octopine-type” Agrobacteria; and Agrobacterium strains which cause production of agropine (e.g., strain EHA105, EHA101, A281, etc.) are referred to as “agropine-type” Agrobacteria.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

As used herein, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; typically, one sequence is a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software.

As used herein, the term plant cell “compartments or organelles” is used in its broadest sense. As used herein, the term includes but is not limited to, the endoplasmic reticulum, Golgi apparatus, trans Golgi network, plastids, sarcoplasmic reticulum, glyoxysomes, mitochondrial, chloroplast, thylakoid membranes and nuclear membranes, and the like.

As used herein, the term “trait” in reference to a plant refers to an observable and/measurable characteristics of an organism, such as cold tolerance in a plant or microbe. As used herein, the term “agronomic trait” and “economically significant trait” refers to any selected trait that increases the commercial value of a plant part, for example a preferred yield, a oil content, protein content, seed protein content, seed size, seed color, seed coat thickness, seed sugar content, leaf soluble sugar content, leaf starch content, seed free amino acid content, seed germination rate, seed texture, seed fiber content, food-grade quality, hilum color, seed yield, color of a plant part, drought resistance, water resistance, cold weather resistance, hot weather resistance, and growth in a particular hardiness zone.

As used herein, “aerial” and “aerial parts of Arabidopsis plants” refers to any plant part that is above water in aquatic plants or any part of a terrestrial plant part found above ground level.

The term “variety” refers to a biological classification for an intraspecific group or population, that can be distinguished from the rest of the species by any characteristic (for example morphological, physiological, cytological, etc.). A variety may originate in the wild but can also be produced through selected breeding (for example, see, cultivar).

The terms “cultivar,” “cultivated variety,” and “cv” refer to a group of cultivated plants distinguished by any characteristic (for example morphological, physiological, cytological, etc.) that when reproduced sexually or asexually, retain their distinguishing features to produce a cultivated variety.

The term “propagation” refers to the process of producing new plants, either by vegetative means involving the rooting or grafting of pieces of a plant, or by sowing seeds. The terms “vegetative propagation” and “asexual reproduction” refer to the ability of plants to reproduce without sexual reproduction, by producing new plants from existing vegetative structures that are clones, i.e., plants that are identical in all attributes to the mother plant and to one another. For example, the division of a clump, rooting of proliferations, or cutting of mature crowns can produce a new plant.

The terms “tissue culture” and “micropropagation” refer to a form of asexual propagation undertaken in specialized laboratories, in which clones of plants are produced from small cell clusters from very small plant parts (e.g. buds, nodes, leaf segments, root segments, etc.), grown aseptically (free from any microorganism) in a container where the environment and nutrition can be controlled.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. The term “a nucleotide comprising at least a portion of a gene” may comprise fragments of the gene or the entire gene. The term “cDNA” refers to a nucleotide copy of the “messenger RNA” or “mRNA” for a gene. In some embodiments, cDNA is derived from the mRNA. In some embodiments, cDNA is derived from genomic sequences. In some embodiments, cDNA is derived from EST sequences. In some embodiments, cDNA is derived from assembling portions of coding regions extracted from a variety of BACs, contigs, Scaffolds and the like.

The term “gene” encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences.

The term “gene” encompasses both cDNA and genomic forms of a gene. A genomic form or clone of a gene contains the coding region termed “exon” or “expressed regions” or “expressed sequences” interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage and polyadenylation.

The term “heterologous” when used in reference to a gene or nucleic acid refers to a gene that has been manipulated in some way. For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed).

The terms “nucleic acid sequence,” “nucleotide sequence of interest” or “nucleic acid sequence of interest” refer to any nucleotide sequence (e.g., RNA or DNA), the manipulation of which may be deemed desirable for any reason (e.g., treat disease, confer improved qualities, etc.), by one of ordinary skill in the art. Such nucleotide sequences include, but are not limited to, coding sequences of structural genes (e.g., reporter genes, selection marker genes, oncogenes, drug resistance genes, growth factors, etc.), and non-coding regulatory sequences which do not encode an mRNA or protein product (e.g., promoter sequence, polyadenylation sequence, termination sequence, enhancer sequence, etc.).

The term “oligonucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof.

The term “polynucleotide” refers to refers to a molecule comprised of several deoxyribonucleotides or ribonucleotides, and is used interchangeably with oligonucleotide. Typically, oligonucleotide refers to shorter lengths, and polynucleotide refers to longer lengths, of nucleic acid sequences.

The term “an oligonucleotide (or polypeptide) having a nucleotide sequence encoding a gene” or “a nucleic acid sequence encoding” a specified polypeptide refers to a nucleic acid sequence comprising the coding region of a gene or in other words the nucleic acid sequence which encodes a gene product. The coding region may be present in a cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc., may be placed in close proximity to the coding region of the gene if needed to permit proper initiation of transcription and/or correct processing of the primary RNA transcript. Alternatively, the coding region utilized in the expression vectors of the present invention may contain endogenous enhancers, exogenous promoters, splice junctions, intervening sequences, polyadenylation signals, etc., or a combination of both endogenous and exogenous control elements.

As used herein, the term “exogenous promoter” refers to a promoter in operable combination with a coding region wherein the promoter is not the promoter naturally associated with the coding region in the genome of an organism. The promoter which is naturally associated or linked to a coding region in the genome is referred to as the “endogenous promoter” for that coding region.

The terms “complementary” and “complementarity” refer to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “protein,” “polypeptide,” “peptide,” “encoded product,” “amino acid sequence,” are used interchangeably to refer to compounds comprising amino acids joined via peptide bonds and a “protein” encoded by a gene is not limited to the amino acid sequence encoded by the gene, but includes post-translational modifications of the protein. Where the term “amino acid sequence” is recited herein to refer to an amino acid sequence of a protein molecule, the term “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. The deduced amino acid sequence from a coding nucleic acid sequence includes sequences which are derived from the deduced amino acid sequence and modified by post-translational processing, where modifications include but not limited to glycosylation, hydroxylations, phosphorylations, and amino acid deletions, substitutions, and additions. Thus, an amino acid sequence comprising a deduced amino acid sequence is understood to include post-translational modifications of the encoded and deduced amino acid sequence. The term “X” may represent any amino acid.

The terms “homolog,” “homologue,” “homologous,” and “homology” when used in reference to amino acid sequence or nucleic acid sequence or a protein or a polypeptide refers to a degree of sequence identity to a given sequence, or to a degree of similarity between conserved regions, or to a degree of similarity between three-dimensional structures or to a degree of similarity between the active site, or to a degree of similarity between the mechanism of action, or to a degree of similarity between functions. In some embodiments, a homologue has a greater than 30% sequence identity to a given sequence. In some embodiments, a homologue has a greater than 40% sequence identity to a given sequence. In some embodiments, a homologue has a greater than 60% sequence identity to a given sequence. In some embodiments, a homologue has a greater than 70% sequence identity to a given sequence. In some embodiments, a homologue has a greater than 90% sequence identity to a given sequence. In some embodiments, a homologue has a greater than 95% sequence identity to a given sequence. In some embodiments, homology is determined by comparing internal conserved sequences to a given sequence. In some embodiments, homology is determined by comparing designated conserved functional and/or structural regions, for example a RING domain, a low complexity region or a transmembrane region.

The term “sequence identity” means that two polynucleotide or two polypeptide sequences are identical (i.e., on a nucleotide-by-nucleotide basis or amino acid basis) over the window of comparison. The term “percentage of sequence identity” is calculated by comparing two optimally aligned sequences over the window of comparison, determining the number of positions at which the identical nucleic acid base (e.g., A, T, C, G, U, or I) or amino acid, in which often conserved amino acids are taken into account, occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present.

The term “partially homologous nucleic acid sequence” refers to a sequence that at least partially inhibits (or competes with) a completely complementary sequence from hybridizing to a target nucleic acid and is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a sequence that is completely complementary to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target which lacks even a partial-degree of identity (e.g., less than about 30% identity); in the absence of non-specific binding the probe will not hybridize to the second non-identical target.

The term “substantially homologous” when used in reference to a double-stranded nucleic acid sequence such as a cDNA or genomic clone refers to any probe that can hybridize to either or both strands of the double-stranded nucleic acid sequence under conditions of low to high stringency as described above.

The term “substantially homologous” when used in reference to a single-stranded nucleic acid sequence refers to any probe that can hybridize (i.e., it is the complement of) the single-stranded nucleic acid sequence under conditions of low to high stringency as described above.

The term “expression” when used in reference to a nucleic acid sequence, such as a gene, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (as when a gene encodes a protein), through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “vector” refers to nucleic acid molecules that transfer DNA segment(s). Transfer can be into a cell, cell to cell, etc. The term “vehicle” is sometimes used interchangeably with “vector.”

The terms “expression vector” or “expression cassette” refer to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. The term “expression vector” when used in reference to a construct refers to an expression vector construct comprising, for example, a heterologous DNA encoding a gene of interest and the various regulatory elements that facilitate the production of the particular protein of interest in the target cells. In certain embodiments of the present invention, a nucleic acid sequence of the present invention within an expression vector is operatively linked to an appropriate expression control sequence(s) (promoter) to direct mRNA synthesis.

The terms “in operable combination,” “in operable order,” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, and the like.

Transcriptional control signals in eukaryotes comprise “promoter” and “enhancer” elements. Promoters and enhancers consist of short arrays of DNA sequences that interact specifically with cellular proteins involved in transcription (Maniatis et al., 1987, Science 236:1237; herein incorporated by reference). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types.

The terms “promoter element,” “promoter,” or “promoter sequence” refer to a DNA sequence that is located at the 5′ end (i.e. precedes) of the coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “regulatory region” refers to a gene's 5′ transcribed but untranslated regions, located immediately downstream from the promoter and ending just prior to the translational start of the gene.

The term “promoter region” refers to the region immediately upstream of the coding region of a DNA polymer, and is typically between about 500 bp and 4 kb in length, and is preferably about 1 to 1.5 kb in length. Promoters may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue (e.g., leaves). Tissue specificity of a promoter may be evaluated by, for example, operably linking a reporter gene and/or A reporter gene expressing a reporter molecule, to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be “constitutive” or “inducible.” The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098, herein incorporated by reference), and ubi3 promoters (see e.g., Garbarino and Belknap, 1994, Plant Mol. Biol. 24:119-127, herein incorporated by reference). Such promoters have been used successfully to direct the expression of heterologous nucleic acid sequences in transformed plant tissue.

In contrast, an “inducible” promoter is one that is capable of directing a level of transcription of an operably linked nucleic acid sequence in the presence of a stimulus (e.g., heat shock, chemicals, light, etc.) that is different from the level of transcription of the operably linked nucleic acid sequence in the absence of the stimulus.

The term “regulatory element” refers to a genetic element that controls some aspect of the expression of nucleic acid sequence(s). For example, a promoter is a regulatory element that facilitates the initiation of transcription of an operably linked coding region. Other regulatory elements are splicing signals, polyadenylation signals, termination signals, and the like.

The term “naturally linked” or “naturally located” when used in reference to the relative positions of nucleic acid sequences means that the nucleic acid sequences exist in nature in the relative positions.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al. Molecular Cloning: A Laboratory Manual, 2.sup.nd ed., Cold Spring Harbor Laboratory Press, New York (1989) pp. 16.7-16.8, herein incorporated by reference). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, viral infection, biolistics (i.e., particle bombardment) and the like.

The terms “stable transfection” and “stably transfected” refer to the introduction and integration of foreign DNA into the genome of the transfected cell. The term “stable transfectant” refers to a cell that has stably integrated foreign DNA into the genomic DNA.

The terms “transient transfection” and “transiently transfected” refer to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb in Virol., 52:456 (1973), herein incorporated by reference, has been modified by several groups to optimize conditions for particular types of cells. The art is well aware of these numerous modifications.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The terms “bombarding, “bombardment, and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, herein incorporated by reference), and are commercially available (e.g. the helium gas-driven microprojectile accelerator (PDS-1000/He, BioRad).

The term “microwounding” when made in reference to plant tissue refers to the introduction of microscopic wounds in that tissue. Microwounding may be achieved by, for example, particle bombardment as described herein.

The term “overexpression” generally refers to the production of a gene product in transgenic organisms that exceeds levels of production in normal or non-transformed organisms.

The terms “overexpression” and “overexpressing” and grammatical equivalents, are specifically used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis.

### DETAILED DESCRIPTION OF THE INVENTION

The present invention relates to the production of biodiesel. In particular, the present invention provides systems and methods for fermenting biomass materials with transgenic plant materials expressing the WRI1 transcription factor.

Plant oils are the most energy rich biofuel available from plants and can be extracted or extruded from crops with low energy inputs. The main limitation to expanded use of plant oils as petroleum replacements is the lower oil yields per acre of most oilseed crops. To move forward toward large scale crop-based biodiesel production systems will require genetic reprogramming of plants to accumulate large amounts or oil at the proper stage of growth so that maximum oil per acre is obtained.

Plants that accumulate oil in leaves and roots have been produced by transgenic modifications. In particular, the WRI1 transcription factor of Arabidopsis controls primary metabolism in seeds and is required for seed oil biosynthesis. Cernac et al., Plant Physiol. 141:745-57 (2006); Cernac and Benning, Plant J. 40:575-85 (2004); Focks and Benning, Plant Physiol. 118:91-101 (1998); Ruuska et al., Plant Cell 14:1191-1206 (2002).

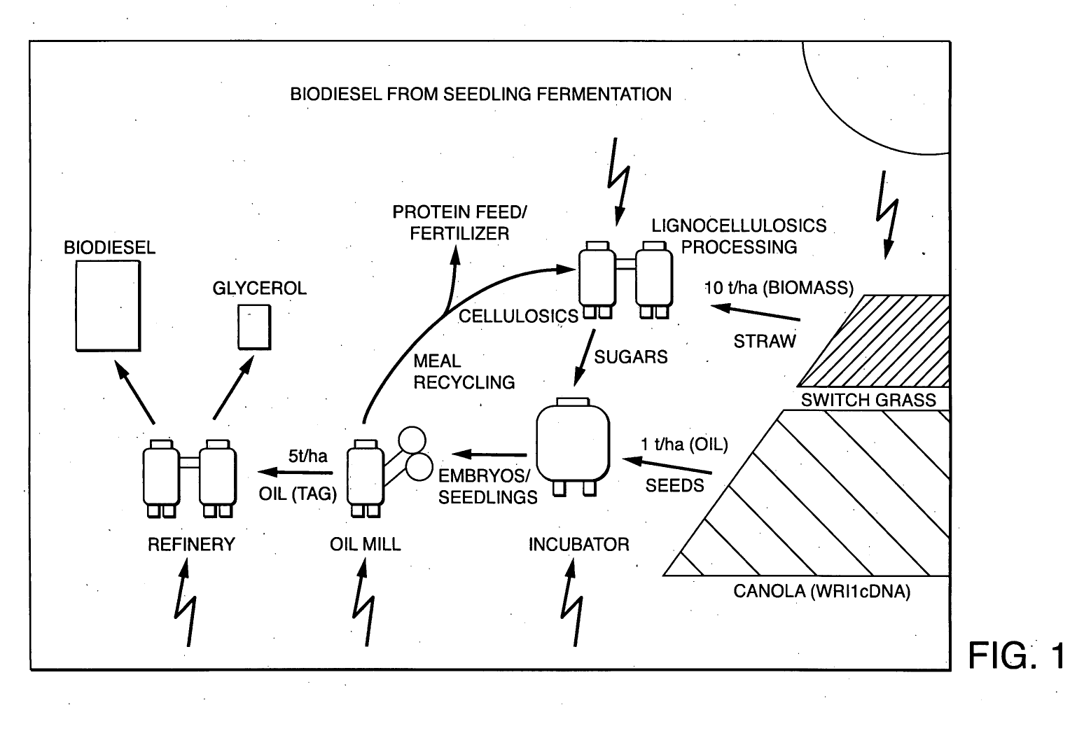

The present invention provides novel methods, compositions, and systems for the production of biodiesel. As shown in FIG. 1, energy from the sun is used to produce a biomass material and plants that express exogenous WRI1. The biomass material is preferably subjected lignocellulosic processing to release sugars from the biomass materials. These sugars are then combined with seeds or seedlings that express exogenous WRI1. The sugars and seeds/seedlings are incubated or fermented so that plant triacylglycerols are produced using the sugars from the biomass. The seed/seedling/biomass sugar feedstock is then milled. The milling process produces triacylglycerols that can further be refined into desired products such as biodiesel. Meal is produced as a by-product which can be used as feed or fertilizer or which can be used as a source of cellulosic materials in the lignocellulosic processing step. The individual components of this system are described in more detail below.

### 1. Sources of WRI1 Activity

In some embodiments of the present invention, plant material expressing the WRI1 transcription factor is contacted with biomass materials so that the plant material produces triacylglycerols. The present invention is not limited to the use of any particular plant materials expressing the WRI1 transcription factor. Indeed, the use of a variety of plant materials is contemplated, including seeds, seedlings, leaves, stems, fruit, roots and the like. In particular preferred embodiments, seeds, germinated seeds, and seedlings are utilized. Likewise, the present invention is not limited to the use of any particular species of plant. Indeed, the use of a variety of plants is contemplated, including, but not limited to, soybean (Glycine max), rapeseed and canola (including Brassica napus and B. campestris), sunflower (Helianthus annus), cotton (Gossypium hirsutum), corn (Zea mays), cocoa (Theobroma cacao), safflower (Carthamus tinctorius), oil palm (Elaeis guineensis), coconut palm (Cocos nucifera), flax (Linum usitatissimum), castor (Ricinus communis) and peanut (Arachis hypogaea).

In preferred embodiments, the plant material comprises an exogenous WRI1 gene. The present invention is not limited to a particular WRI1 gene sequence. Exemplary sequences are described in U.S. Pat. Appl. No. 20030097685, incorporated herein by reference in its entirety. In some preferred embodiments, the WRI1 sequence is at least 65%, 70%, 80%, 90% or 95% identical to SEQ ID NO:1.

The methods of the present invention contemplate the use of at least one heterologous gene encoding a WRI1 gene. Heterologous genes intended for expression in plants are first assembled in expression cassettes comprising a promoter. Methods which are well known to those skilled in the art may be used to construct expression vectors containing a heterologous gene and appropriate transcriptional and translational control elements. These methods include in vitro recombinant DNA techniques, synthetic techniques, and in vivo genetic recombination. Such techniques are widely described in the art (See e.g., Sambrook. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.).

In general, these vectors comprise a nucleic acid sequence of the invention encoding a WRI1 gene of the present invention (as described above) operably linked to a promoter and other regulatory sequences (e.g., enhancers, polyadenylation signals, etc.) required for expression in a plant.

Promoters include but are not limited to constitutive promoters, tissue-, organ-, and developmentally-specific promoters, and inducible promoters. Examples of promoters include but are not limited to: constitutive promoter 35S of cauliflower mosaic virus; the Universal Seed Promoter (USP) from Vicia faba; seed specific promoters from Arabidopsis thaliana, including 2S Seed Storage Protein 1 and 3 Precursor promoter (Accession No. AL035680); 12S Cruciferin promoter (Accession No. AL021749) and vicilin promoter (Accession No. AB022223); a wound-inducible promoter from tomato, leucine amino peptidase (“LAP,” Chao et al. (1999) Plant Physiol 120: 979-992); a chemically-inducible promoter from tobacco, Pathogenesis-Related 1 (PR1) (induced by salicylic acid and BTH (benzothiadiazole-7-carbothioic acid S-methyl ester)); a tomato proteinase inhibitor II promoter (PIN2) or LAP promoter (both inducible with methyl jasmonate); a heat shock promoter (U.S. Pat. No. 5,187,267); a tetracycline-inducible promoter (U.S. Pat. No. 5,057,422); and seed-specific promoters, such as those for seed storage proteins (e.g., phaseolin, napin, oleosin, and a promoter for soybean beta conglycin (Beachy et al. (1985) EMBO J. 4: 3047-3053)). In some preferred embodiments, the promoter is a phaseolin promoter. All references cited herein are incorporated in their entirety.

The expression cassettes may further comprise any sequences required for expression of mRNA. Such sequences include, but are not limited to transcription terminators, enhancers such as introns, viral sequences, and sequences intended for the targeting of the gene product to specific organelles and cell compartments.

A variety of transcriptional terminators are available for use in expression of sequences using the promoters of the present invention. Transcriptional terminators are responsible for the termination of transcription beyond the transcript and its correct polyadenylation. Appropriate transcriptional terminators and those which are known to function in plants include, but are not limited to, the CaMV 35S terminator, the tml terminator, the pea rbcS E9 terminator, and the nopaline and octopine synthase terminator (See e.g., Odell et al. (1985) Nature 313:810; Rosenberg et al. (1987) Gene, 56:125; Guerineau et al. (1991) Mol. Gen. Genet., 262:141; Proudfoot (1991) Cell, 64:671; Sanfacon et al. Genes Dev., 5:141; Mogen et al. (1990) Plant Cell, 2:1261; Munroe et al. (1990) Gene, 91:151; Ballad et al. (1989) Nucleic Acids Res. 17:7891; Joshi et al. (1987) Nucleic Acid Res., 15:9627).

In addition, in some embodiments, constructs for expression of the gene of interest include one or more of sequences found to enhance gene expression from within the transcriptional unit. These sequences can be used in conjunction with the nucleic acid sequence of interest to increase expression in plants. Various intron sequences have been shown to enhance expression, particularly in monocotyledonous cells. For example, the introns of the maize Adh1 gene have been found to significantly enhance the expression of the wild-type gene under its cognate promoter when introduced into maize cells (Calais et al. (1987) Genes Develop. 1: 1183). Intron sequences have been routinely incorporated into plant transformation vectors, typically within the non-translated leader.

In some embodiments of the present invention, the construct for expression of the nucleic acid sequence of interest also includes a regulator such as a nuclear localization signal (Calderone et al. (1984) Cell 39:499; Lassoer et al. (1991) Plant Molecular Biology 17:229), a plant translational consensus sequence (Joshi (1987) Nucleic Acids Research 15:6643), an intron (Luehrsen and Walbot (1991) Mol. Gen. Genet. 225:81), and the like, operably linked to the nucleic acid sequence encoding a polypeptide that inhibits tocopherol biosynthesis.

In preparing a construct comprising a nucleic acid sequence encoding a WRI1 gene of the present invention, various DNA fragments can be manipulated, so as to provide for the DNA sequences in the desired orientation (e.g., sense or antisense) orientation. For example, adapters or linkers can be employed to join the DNA fragments or other manipulations can be used to provide for convenient restriction sites, removal of superfluous DNA, removal of restriction sites, or the like. For this purpose, in vitro mutagenesis, primer repair, restriction, annealing, resection, ligation, or the like is preferably employed, where insertions, deletions or substitutions (e.g., transitions and transversions) are involved.

Numerous transformation vectors are available for plant transformation. The selection of a vector for use will depend upon the preferred transformation technique and the target species for transformation. For certain target species, different antibiotic or herbicide selection markers are preferred. Selection markers used routinely in transformation include the nptII gene which confers resistance to kanamycin and related antibiotics (Messing and Vierra (1982) Gene 19: 259; Bevan et al. (1983) Nature 304:184), the bar gene which confers resistance to the herbicide phosphinothricin (White et al. (1990) Nucl Acids Res. 18:1062; Spencer et al. (1990) Theor. Appl. Genet. 79:625), the hph gene which confers resistance to the antibiotic hygromycin (Blochlinger and Diggelmann (1984) Mol. Cell. Biol. 4:2929), and the dhfr gene, which confers resistance to methotrexate (Bourouis et al. (1983) EMBO J., 2:1099).

In some preferred embodiments, the vector is adapted for use in an Agrobacterium mediated transfection process (See e.g., U.S. Pat. Nos. 5,981,839; 6,051,757; 5,981,840; 5,824,877; and 4,940,838; all of which are incorporated herein by reference). Construction of recombinant Ti and Ri plasmids in general follows methods typically used with the more common bacterial vectors, such as pBR322. Additional use can be made of accessory genetic elements sometimes found with the native plasmids and sometimes constructed from foreign sequences. These may include but are not limited to structural genes for antibiotic resistance as selection genes.

There are two systems of recombinant Ti and Ri plasmid vector systems now in use. The first system is called the “cointegrate” system. In this system, the shuttle vector containing the gene of interest is inserted by genetic recombination into a non-oncogenic Ti plasmid that contains both the cis-acting and trans-acting elements required for plant transformation as, for example, in the pMLJ1 shuttle vector and the non-oncogenic Ti plasmid pGV3850. The second system is called the “binary” system in which two plasmids are used; the gene of interest is inserted into a shuttle vector containing the cis-acting elements required for plant transformation. The other necessary functions are provided in trans by the non-oncogenic Ti plasmid as exemplified by the pBIN19 shuttle vector and the non-oncogenic Ti plasmid PAL4404. Some of these vectors are commercially available.

In other embodiments of the invention, the nucleic acid sequence of interest is targeted to a particular locus on the plant genome. Site-directed integration of the nucleic acid sequence of interest into the plant cell genome may be achieved by, for example, homologous recombination using Agrobacterium-derived sequences. Generally, plant cells are incubated with a strain of Agrobacterium which contains a targeting vector in which sequences that are homologous to a DNA sequence inside the target locus are flanked by Agrobacterium transfer-DNA (T-DNA) sequences, as previously described (U.S. Pat. No. 5,501,967). One of skill in the art knows that homologous recombination may be achieved using targeting vectors which contain sequences that are homologous to any part of the targeted plant gene, whether belonging to the regulatory elements of the gene, or the coding regions of the gene. Homologous recombination may be achieved at any region of a plant gene so long as the nucleic acid sequence of regions flanking the site to be targeted is known.

In yet other embodiments, the nucleic acids of the present invention are utilized to construct vectors derived from plant (+) RNA viruses (e.g., brome mosaic virus, tobacco mosaic virus, alfalfa mosaic virus, cucumber mosaic virus, tomato mosaic virus, and combinations and hybrids thereof). Generally, the inserted polypeptide that inhibits tocopherol biosynthesis) can be expressed from these vectors as a fusion protein (e.g., coat protein fusion protein) or from its own subgenomic promoter or other promoter. Methods for the construction and use of such viruses are described in U.S. Pat. Nos. 5,846,795; 5,500,360; 5,173,410; 5,965,794; 5,977,438; and 5,866,785, all of which are incorporated herein by reference.

In some embodiments of the present invention the nucleic acid sequence of interest is introduced directly into a plant. One vector useful for direct gene transfer techniques in combination with selection by the herbicide Basta (or phosphinothricin) is a modified version of the plasmid pCIB246, with a CaMV 35S promoter in operational fusion to the E. coli GUS gene and the CaMV 35S transcriptional terminator (WO 93/07278).

Once a nucleic acid sequence encoding WRI1 is operatively linked to an appropriate promoter and inserted into a suitable vector for the particular transformation technique utilized (e.g., one of the vectors described above), the recombinant DNA described above can be introduced into the plant cell in a number of art-recognized ways. Those skilled in the art will appreciate that the choice of method might depend on the type of plant targeted for transformation. In some embodiments, the vector is maintained episomally. In other embodiments, the vector is integrated into the genome.

In some embodiments, the vector is introduced through ballistic particle acceleration using devices (e.g., available from Agracetus, Inc., Madison, Wis. and Dupont, Inc., Wilmington, Del.). (See e.g., U.S. Pat. No. 4,945,050; and McCabe et al. (1988) Biotechnology 6:923). See also, Weissinger et al. (1988) Annual Rev. Genet. 22:421; Sanford et al. (1987) Particulate Science and Technology, 5:27 (onion); Svab et al. (1990) Proc. Natl. Acad. Sci. USA, 87:8526 (tobacco chloroplast); Christou et al. (1988) Plant Physiol., 87:671 (soybean); McCabe et al. (1988) Bio/Technology 6:923 (soybean); Klein et al. (1988) Proc. Natl. Acad. Sci. USA, 85:4305 (maize); Klein et al. (1988) Bio/Technology, 6:559 (maize); Klein et al. (1988) Plant Physiol., 91:4404 (maize); Fromm et al. (1990) Bio/Technology, 8:833; and Gordon-Kamm et al. (1990) Plant Cell, 2:603 (maize); Koziel et al. (1993) Biotechnology, 11:194 (maize); Hill et al. (1995) Euphytica, 85:119 and Koziel et al. (1996) Annals of the New York Academy of Sciences 792:164; Shimamoto et al. (1989) Nature 338: 274 (rice); Christou et al. (1991) Biotechnology, 9:957 (rice); Datta et al. (1990) Bio/Technology 8:736 (rice); European Patent Application EP 0 332 581 (orchardgrass and other Pooideae); Vasil et al. (1993) Biotechnology, 11: 1553 (wheat); Weeks et al. (1993) Plant Physiol., 102: 1077 (wheat); Wan et al. (1994) Plant Physiol. 104: 37 (barley); Jahne et al. (1994) Theor. Appl. Genet. 89:525 (barley); Knudsen and Muller (1991) Planta, 185:330 (barley); Umbeck et al. (1987) Bio/Technology 5: 263 (cotton); Casas et al. (1993) Proc. Natl. Acad. Sci. USA 90:11212 (sorghum); Somers et al. (1992) Bio/Technology 10:1589 (oat); Torbert et al. (1995) Plant Cell Reports, 14:635 (oat); Weeks et al. (1993) Plant Physiol., 102:1077 (wheat); Chang et al., WO 94/13822 (wheat) and Nehra et al. (1994) The Plant Journal, 5:285 (wheat).

In other embodiments, direct transformation in the plastid genome is used to introduce the vector into the plant cell (See e.g., U.S. Pat. Nos. 5,451,513; 5,545,817; 5,545,818; PCT application WO 95/16783). The basic technique for chloroplast transformation involves introducing regions of cloned plastid DNA flanking a selectable marker together with the nucleic acid encoding the RNA sequences of interest into a suitable target tissue (e.g., using biolistics or protoplast transformation with calcium chloride or PEG). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al. (1990) PNAS, 87:8526; Staub and Maliga, (1992) Plant Cell, 4:39). The presence of cloning sites between these markers allowed creation of a plastid targeting vector introduction of foreign DNA molecules (Staub and Maliga (1993) EMBO J., 12:601). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab and Maliga (1993) PNAS, 90:913). Other selectable markers useful for plastid transformation are known in the art and encompassed within the scope of the present invention. Plants homoplasmic for plastid genomes containing the two nucleic acid sequences separated by a promoter of the present invention are obtained, and are preferentially capable of high expression of the RNAs encoded by the DNA molecule.

In other embodiments, vectors useful in the practice of the present invention are microinjected directly into plant cells by use of micropipettes to mechanically transfer the recombinant DNA (Crossway (1985) Mol. Gen. Genet, 202:179). In still other embodiments, the vector is transferred into the plant cell by using polyethylene glycol (Krens et al. (1982) Nature, 296:72; Crossway et al. (1986) BioTechniques, 4:320); fusion of protoplasts with other entities, either minicells, cells, lysosomes or other fusible lipid-surfaced bodies (Fraley et al. (1982) Proc. Natl. Acad. Sci., USA, 79:1859); protoplast transformation (EP 0 292 435); direct gene transfer (Paszkowski et al. (1984) EMBO J., 3:2717; Hayashimoto et al. (1990) Plant Physiol. 93:857).

In still further embodiments, the vector may also be introduced into the plant cells by electroporation (Fromm, et al. (1985) Proc. Natl. Acad. Sci. USA 82:5824; Riggs et al. (1986) Proc. Natl. Acad. Sci. USA 83:5602). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

In addition to direct transformation, in some embodiments, the vectors comprising a nucleic acid sequence encoding a WRI1 gene of the present invention are transferred using Agrobacterium-mediated transformation (Hinchee et al. (1988) Biotechnology, 6:915; Ishida et al. (1996) Nature Biotechnology 14:745). Agrobacterium is a representative genus of the gram-negative family Rhizobiaceae. Its species are responsible for plant tumors such as crown gall and hairy root disease. In the dedifferentiated tissue characteristic of the tumors, amino acid derivatives known as opines are produced and catabolized. The bacterial genes responsible for expression of opines, are a convenient source of control elements for chimeric expression cassettes. Heterologous genetic sequences (e.g., nucleic acid sequences operatively linked to a promoter of the present invention), can be introduced into appropriate plant cells, by means of the Ti plasmid of Agrobacterium tumefaciens. The Ti plasmid is transmitted to plant cells on infection by Agrobacterium tumefaciens, and is stably integrated into the plant genome (Schell (1987) Science, 237: 1176). Species which are susceptible to infection by Agrobacterium may be transformed in vitro. Alternatively, plants may be transformed in vivo, such as by transformation of a whole plant by Agrobacteria infiltration of adult plants, as in a “floral dip” method (Bechtold N, Ellis J, Pelletier G (1993) Cr. Acad. Sci. III—Vie 316: 1194-1199).

After selecting for transformed plant material that can express the heterologous gene encoding a WRI1 gene of the present invention, whole plants are regenerated. Plant regeneration from cultured protoplasts is described in Evans et al. (1983) Handbook of Plant Cell Cultures, Vol. 1: (MacMillan Publishing Co. New York); and Vasil I. R. (ed.), Cell Culture and Somatic Cell Genetics of Plants, Acad. Press, Orlando, Vol. 1 (1984), and Vol. III (1986). It is known that many plants can be regenerated from cultured cells or tissues, including but not limited to all major species of crop plants, Arabidopsis, sugarcane, sugar beet, cotton, fruit and other trees, legumes and vegetables, and monocots (e.g., the plants described above). Means for regeneration vary from species to species of plants, but generally a suspension of transformed protoplasts containing copies of the heterologous gene is first provided. Callus tissue is formed and shoots may be induced from callus and subsequently rooted.

Alternatively, embryo formation can be induced from the protoplast suspension. These embryos germinate and form mature plants. The culture media will generally contain various amino acids and hormones, such as auxin and cytokinins. Shoots and roots normally develop simultaneously. Efficient regeneration will depend on the medium, on the genotype, and on the history of the culture. The reproducibility of regeneration depends on the control of these variables.

The presence of nucleic acid sequences encoding a WRI1 gene of the present invention (including mutants or variants thereof) may be transferred to related varieties by traditional plant breeding techniques. The transgenic lines are then utilized for generation of biofuels as described herein.

### 2. Sources of Biomass

In preferred embodiments, the seeds or seedlings expressing the exogenous WRI1 gene are combined with a biomass material. The present invention is not limited to the use of any particular biomass material. Indeed, the use of a variety of biomass materials is contemplated. In preferred embodiments, the biomass material is an agricultural biomass material or forest biomass material. In some preferred embodiments, the biomass materials are lignocellulosic biomass materials or starch or sugars derived from lignocellulosic biomass material or crops such as sugarcane, sugar beets, etc. Agricultural biomass materials include, but are not limited to, crops such as corn, wheat, oats, soybeans, sorghum, millet and rice), crops residues such as corn stover and straw from wheat, oats, barley and other small grains, sorghum stover, perennial grasses (such as timothy, (Phleum pratense L.), tall fescue (Festuca arundinacea Schreb.), reed canarygrass (Phalaris arundinacea L.) switchgrass (Panicum virgatum L.), rye (Secale cereale L.), elephantgrass, energycane, sugarcane, and Erianthus), annual grasses (such as sorghum x sudangrass (Sorghum bicolor L. Moench), and two forage sorghum (Sorghum bicolor L. Moench)), perennial woody crops (such a hybrid poplars, hemp, and short rotation coppice), annual woody crops, dry distillers grain, corn residue left after sweetener processing, and manure. Forest biomass material includes, but are not limited to, logging residues from harvest operations such as treetops, limbs, branches and leaves, residues from forest management and clearing operations, primary wood processing mill residues such as bark, course residues (chunks and slabs) and fine residues (shavings and sawdust), secondary wood processing mill residues (such as millwork, containers, pallets, sawdust, sander dust, cut-offs and other scrap wood), and urban wood residues including construction and demolition debris, tree trimmings, and packaging wastes.

### 3. Biomass Processing

In some embodiments, the biomass material, preferably lignocellulosic biomass material is treated to provide sugars. This process is called saccharization. Lignocellosic materials comprise cellulose, hemicellulose and lignin. Cellulose is a polysaccharide comprising glucopyranose subunits joined by β-1→4 glucosidic bonds. The monomer subunits are glucose. Hemicellulose are groups of polysaccharides including four basic types: D-xyloglucans, D-xylans, D-mannans, and D-galactans. In each type, two to six monomers are linked by β-1→4 and β-1→3 bonds in main chained and α-1→2, 3, and 6 binds in branches. The monomer subunits can be D-xylose, L-arabinose, D-mannose, D-glucose, D-galactose, and D-glucouronic acid. Core lignins are highly condensed polymers formed by dehydrogenative polymerization of the hydroxycinnamyl alcohols, p-coumaryl alcohols, coniferyl alcohols, and sinapyl alcohols. Non-core lignin includes esterified or etherified phenolic acids bound to core lignin or noncellulosic polysaccharides. In preferred embodiments, the biomass material comprising cellulose, hemicelluose and lignin (i.e., lignocellulosic biomass) is treated to produce glucose, fructose, sucrose, mannose, maltose, sorbitol, galactose, xylose, and combinations thereof. In preferred embodiments, lignocellulosic biomass materials are hydrolyzed. The present invention is not limited to the use of any particular hydrolysis method. Indeed, the use of a variety of hydrolysis methods are contemplated, including, but not limited to, enzymatic hydrolysis and chemical hydrolysis (such as dilute acid hydrolysis or concentrated acid hydrolysis) and combinations thereof.

In some preferred embodiments, the biomass material chemically hydrolyzed. In some embodiments, the biomass material is treated with an acid solution, such as hydrochloric acid solution or sulfuric acid solution. In some embodiments, the solution comprises about, 10, 20, 30, 40, 50, 60, 70, 75, 80, or 85 percent acid, preferably sulfuric acid.

In other embodiments, the biomass material is enzymatically hydrolyzed. In some embodiments, the biomass materials are treated with enzymes that hydrolyze cellulose (i.e., a cellulose) and/or hemicellulose (i.e., a hemicellulase). Examples of commercially available enzymes useful in the present invention include, but are not limited to Spezyme CP (Genencor), β-glucosidase (Novozyme). Other useful enzymes include, but are not limited to, carboxymethyl cellulose (endoglycanase), Maize-all®, Cellulast®, Viscozyme®, cellbiase, xylanase, amylase, pectinase, cellobiohydralase (exoglucanase). In general, useful enzymes are isolated from the following cellulolytic fungi: Acremonium cellulolyticus, Aspergillus acculeatus, Aspergillus fumigatus, Aspergillis niger, Fusarium solani, Irpex lacteus, Penicillium funmiculosum, Phanerachaete, Cchrysosporium, Schizophyllum commune, Sclerotium relfsii, Sporottichum cellulophilum, Talaromycees emersonii, Thielevia terrestris, Trichoderma koningii, Trichoderma reesei, and Thrichoderma viride. Useful enzymes are also isolated from the following bacteria: Clostridium thermocellum, Ruminococcus albus, and Streptomycees. In some embodiments, purified enzymes are used to treat the biomass material. In other embodiments, the biomass material is inoculated with a culture of one or more the foregoing organism and incubated to allow degradation of the biomass material.

In some embodiments, the biomass material is pretreated prior to chemical and/or enzymatic hydrolysis. In some preferred embodiments, the biomass material is pretreated by uncatalyzed steam explosion, liquid hot water (200° C., 20-24 atm, 24 minutes), pH controlled hot water (170-200° C., 6-14 atm, 5-20 minutes), flow-through liquid hot water, dilute acid (0.22-0.98% sulfuric acid at 140-200° C., 3-15 atm, 2-30 minutes) flow-through acid, ammonia fiber/freeze explosion (100% anhydrous ammonia, 60-110° C., 15-20 atm, 5 minutes), ammonia recycle percolation (10-15 wt. % ammonia, 110-170° C., 9-17 atm, 10-20 minutes), lime pretreatment (0.5 g Ca(OH)₂/g biomass, 25-55° C., 1-6 atm, 4 weeks), or combination thereof.

4. Culture of Plant Material with Biomass Material

In some embodiments, the biomass material is combined with plant material comprising an exogenous WRI1 gene. In some preferred embodiments, the plant material comprising an exogenous WRI1 gene is a seed, germinated seed, or seedling. In still further preferred embodiments, the biomass material is lignocellulosic biomass material that has been enzymatically or chemically treated as described above or sugars and starch from crops such as sugarcane or sugar beets.

In some preferred embodiments, seeds are combined with the treated biomass material in a seedling fermentation process. The present invention is not limited to any particular mechanism of action. Indeed, an understanding of the mechanism of action is not necessary to practice the present invention. In some preferred embodiments, the seeds germinate. Following germination, the expression of WRI1 activates pathways for the synthesis of plant triacylglycerols using sugars derived from saccharization of the biomass material or otherwise produced sugars. The germinated seeds develop into seedlings that accumulate plant triacylglycerols which can then be extracted.

In some preferred embodiments, the seedling fermentation process utilizes liquid culture. In these embodiments, the seeds and treated biomass materials are combined in an aqueous environment. In other embodiments, the seeds are cultured on a screen that is periodic wetted with a solution comprising the extracted biomass material. In still further preferred embodiments, the seeds are cultured on wetted substrate such as paper and periodically treated with a solution comprising the treated biomass material. In some embodiments, the culture systems are exposed to light. However, in other embodiments, the culture systems are maintained in the dark or with red light.

### 5. Uses of Plant Triacylgycerols

The triacylgycerols produced by the methods described have a variety of uses. In some embodiments, the triacylgycerols are used as food oils. In other embodiments, the triacyglycerols are refined and used as lubricants or for other industrial uses such as the synthesis of plastics.

In some preferred embodiments, the triacylglycerols are refined to produce biodiesel. In some preferred embodiments, the triacylglycerols are transesterified to produce methyl esters and glycerol. In some preferred embodiments, the triacyglycerols are reacted with an alcohol (such as methanol or ethanol) in the presence of a catalyst (potassium or sodium hydroxide) the produce alkylesters. The alkylesters can be used for biodiesel or blended with petroleum based fuels.

### Experimental

### Optimization of Growth Surfaces

The following growth systems for optimal Seedling Fermentation will be evaluated: liquid culture, on screens periodically wetted with nutrient solution, and on wetted paper based material with media compositions mimicking those used for agar preparation. Testing these different growth surfaces may provide valuable information that should enhance Seedling Fermentation optimization efforts. For example, seedling germination in liquid culture may enhance free access to sugars in the medium and positively impact fermentation efficiency. In contrast, liquid culture may be disruptive to growth of the seedling and negatively impact fermentation efficiency, thus growth on screens or wetted paper may be preferred as it should provide free access to media sugars, but not involve mechanical agitation. In addition, the use of liquid, screen or paper based stratum may reduce sugar concentration requirements for observed Seedling Fermentation.

### Nutrient Optimization

Nutrient sugars provided in the growth medium, are utilized by the germinating seedling for energy as well as a carbon source for Seedling Fermentation. A variety of nutrient sugars at various concentrations and utilizing several different combinations will be analyzed for successful Seedling Fermentation. Depending on the specificity of the required nutrient sugar composition, it is possible that a very crude lignocellulosic plant extract may be sufficient for fueling the Seedling Fermentation process. Nutrient sugars to be examined include but are not limited to the following: glucose, fructose, sucrose, mannose, maltose, sorbitol, galactose and xylose. In addition the different lignocellulosic fraction of plant extract treated with different hydrolytic enzymes will be examined for utilization in the Seedling Fermentation process alone and in combination with nutrient sugar(s).

### Optimization of Light Conditions:

The embryo-like characteristics of the germinating seedling that apparently contribute to the storage and accumulation of TAG during Seedling Fermentation are expected to be a light-independent process. It is possible that exposure to light activates systems that inhibit Seedling Fermentation, or negatively affect the accumulation of TAG. To address these concerns, Seedling Fermentation will be initiated in both light and dark and also in red light conditions.

### Evaluation of Sterility

Thorough sterilization of seeds reduces germination efficiency, which negatively affects projected TAG production. In addition, stringent sterility requirements directly increase both engineering investment and operational expenses. Current activities incorporate near maximal sterile conditions. At seedlings require minimal growth time while Canola seeds will germinate at much reduced temperatures (ie 4-10° C.). These attributes may provide the opportunity to control or reduce microbial growth with minimal expense, and allow the use of growth medium nutrient sugars that are less than sterile in the Seedling Fermentation process. The use of microbial growth inhibitors will be assessed in terms of operational necessity for the Seedling Fermentation process.

### Evaluation of TAG Production

Seedling Fermentation will result in TAG production in each seedling that often exceeds the amount found in individual wild type seeds by more than 10-fold. Optimization of conditions for Seedling Fermentation is expected to maximize the fold increase in TAG production per seedling. To evaluate the quantity of TAG present in the seedlings, existent lab protocols described previously (Focks and Benning, 1998; Cernac and Benning, 2004) will be utilized. In short, individual seedling TAG composition and quantity will be determined by gas chromatography of fatty acid methyl esters derived from the TAGs. In addition, the composition and quantity of other compounds such as amino acids starch, and sugar and quantities will be evaluated using established protocols (Focks and Benning, 1998; Cernac and Benning, 2004).

### Results

Preliminary Seedling Fermentation results currently indicate that several-fold increases in storage TAG accumulation is possible as compared to TAG that is just present in dry in the seeds. Current optimization strategies are aimed at not only improving the ratio of seedlings that participate in Seedling Fermentation, but also improving the quantity of TAG generated per seedling by maximizing the conversion of the medium provided sugars to TAGs. The type of transgenic line (combination of promoter and strength and timing of expression of the WRI1 transgene), and the type of sugar(s) and availability of the sugar to the seedling are important for efficient Seedling Fermentation. Being able to use crude lignocellulosics fractions or five carbon sugars in the production of TAGs will be a very important improvement over alternative microorganism based fermentation systems. Ensuring the availability of nutrient sugars will be addressed while testing growth media parameters. The immobilization of the seedling on agar may limit the availability of sugar to the seedling. Experimenting with liquid culture and wetted surface based growth surfaces are also expected to provide information related to increasing individual Seedling Fermentation productivity and will provide the basis for further development of an industrial process of Seedling Fermentation. Preliminary experiments suggest that an increased percentage of seedlings participate in the fermentation process when the seedlings are germinated and maintained in the absence of light. Considering the proposed requirement of nutrient sugar as an energy source in the fermentation process, the absence of light may be preferred as light and the establishment of photosynthesis in the developing seedling may act to suppress pathways involved in the storage of seed components. Currently, the needed level of sterility for Seedling Fermentation is not established. Sterility is a concern in the laboratory and will undoubtedly be a much stronger concern in scaled-up scenarios. It is expected that conditions can be worked out that will minimize the necessity for sterility and provide a more practicable up-scaled process.

What is claimed is:

1. A method comprising:
a) providing:
i) first plant material from a first plant comprising an exogenous
WRI1 gene;
ii) lignocellulosic plant material, sugars or starch derived from a second plant;
b) contacting said first plant material with said lignocellulosic plant material under conditions such that triacylglycerols are produced by said first plant material.

2. The method of claim 1, wherein said first plant material is selected from the group consisting of canola, corn, soybean, sunflower and safflower plant material.

3. The method of claim 2, wherein said first plant material is selected from the group consisting of seeds, leaves, germinated seeds, seedlings and combinations thereof.

4. The method of claim 1, wherein said lignocellulosic plant material is selected from the group consisting of perennial grass, annual grass, perennial woody plants, and crop residue.

5. The method of claim 1, wherein said lignocellulosic plant material is treated to hydrolyze cellulose and/or hemicellulose contained in said material.

6. The method of claim 5, wherein said lignocellulosic material is treated by a method selected from the group consisting of chemical and enzymatic treatment.

7. The method of claim 1, wherein said WRI1 gene is at least 70% identical to SEQ ID NO:1.

8. The method of claim 7, wherein said WRI1 gene is operably linked to a promoter selected from the group consisting of 35S CMV promoter, Universal Seed Promotor, 2S Seed Storage Protein Promotor, Cruciferin promoter, and vicilin promoter.

9. The method of claim 1, further comprising the step of extracting said triacylglycerols from said first plant material.

10. The method of claim 9, further comprising the step of refining said triacylglycerols.

11. The method of claim 7, wherein said lignocellulosic material is pretreated prior to said chemical or enzymatic treatment.

12. A method comprising:
a) providing:
i) first plant material from a first plant comprising an exogenous WRI1 gene;
ii) lignocellulosic plant material from a second plant;
b) treating said lignocellulosic plant material to hydrolyze cellulose and hemicellulose to provide hydrolyzed lignocellulosic plant material;
c) contacting said first plant material with said hydrolyzed lignocellulosic plant material under conditions such that triacylglycerols are produced by said first plant material; and
d) extracting said triacylglycerols from said first plant material.

13. A feedstock for a culture process comprising first plant material comprising an exogenous WRI1 gene and hydrolyzed lignocellulosic plant material.

14. The feedstock of claim 13, wherein said first plant material is selected from the group consisting of canola, corn, soybean, sunflower and safflower plant material.

15. The feed stock of claim 14, wherein said first plant material is selected from the group consisting of seeds, leaves, germinated seeds, seedlings and combinations thereof.

16. The feedstock of claim 13, wherein said hydrolyzed lignocellulosic plant material is selected from the group consisting of hydrolyzed perennial grass, annual grass, perennial woody plants, and crop residue.

17. The feedstock of claim 13, wherein said WRI1 gene is at least 70% identical to SEQ ID NO:1.

18. The feedstock of claim 17, wherein said WRI1 gene is operably linked to a promoter selected from the group consisting of 35S CMV promoter, Universal Seed Promotor, 2S Seed Storage Protein Promotor, Cruciferin promoter, and vicilin promoter.

19. A method comprising:
a) providing:
i) first plant material from a first plant comprising an exogenous WRI1 gene;
ii) lignocellulosic plant material from a second plant;
b) treating said lignocellulosic plant material to hydrolyze cellulose and hemicellulose to provide hydrolyzed lignocellulosic plant material;
c) contacting said first plant material with said hydrolyzed lignocellulosic plant material under conditions such that triacylglycerols are produced by said first plant material;
d) extracting said triacylglycerols from said first plant material; and
e) transesterifying said triacylglycerols with an alcohol to produce alkylesters.
